(19) United States
(12) Patent Application Publication
(10) Pub. No.: US 2015/0238118 A1
(43) Pub. Date: Aug. 27, 2015

(54) DETECTION OF THE SPATIAL LOCATION OF AN IMPLANTABLE BIOSENSING PLATFORM AND METHOD THEREOF

(71) Applicant: Biorasis, Inc., Storrs, CT (US)

(72) Inventors: Allen Legassey, Mansfield, CT (US); Robert A. Croce, JR., Guilford, CT (US); Santhisagar Vaddiraju, Willimantic, CT (US); Faquir C. Jain, Storrs, CT (US); Fotios Papadimitrakopoulos, West Hartford, CT (US)

(21) Appl. No.: 14/220,878

(22) Filed: Mar. 20, 2014

Related U.S. Application Data
(60) Provisional application No. 61/945,542, filed on Feb. 27, 2014.

(51) Int. Cl. A61B 5/06; A61B 5/145

(52) U.S. Cl. CPC A61B 5/062; A61B 5/14503; A61B 5/14532

(57) ABSTRACT

A methodology used to pinpoint the location of an implantable biomedical sensing device is provided and is carried out by integrating miniaturized magnets, or materials with magnetic properties into the implantable bio-sensing chip to detect the position of the implant by sensing the induced magnetic field via an external communication unit. Presented here are various configurations in which magnetic positional detection can be carried out. The positional information collected from these detection motifs can be used to provide feedback to the user about alignment status as well as activate a self-alignment methodology. With respect to the former, based on the positional information received the user manually adjusts the location of the external communicator into place to align with the implantable platform. In the latter scenario, various configurations allow the wireless powering and communication components on the proximity communicator to automatically find and align with the implantable biomedical sensing chip.

### CROSS REFERENCE TO RELATED APPLICATIONS

This application claims the benefit of priority of the filing date of U.S. Provisional Patent Application Ser. No. 61/945,542 filed Feb. 27, 2014, the contents of which is incorporated herein by reference in its entirety.

### STATEMENT REGARDING FEDERALLY SPONSORED RESEARCH

The United States Government has certain rights in this invention pursuant to National Institute of Health Grant No. 1R43EB011886 and National Science Foundation Grant No. 1230148.

### FIELD OF THE INVENTION

This invention relates generally to biosensors and more particularly to detecting and locating the position of a biosensor that has been implanted into a subject.

### BACKGROUND OF THE INVENTION

Miniature totally implantable biomedical sensing devices have the potential to diagnose and manage a vast array of physiological ailments due to their ability to deliver continuous, real-time sensor readings, as opposed to data collection at periodic user-defined intervals which does not holistically capture the overall trends in biomarkers of interest (such is the case with finger-prick glucose measurements for diabetes management). It has been shown that continuous monitoring of glucose, lactate, O₂, and other essential physiological metabolites (such as CO₂, glutamate, creatine, etc.) could aid in the prevention and/or diagnosis of many chronic and prevalent diseases. Along these lines, the aforementioned implantable biomedical systems currently under development employ complementary metal-oxide semiconductor (CMOS) fabrication principles integrated with electrochemical sensors in order to achieve the necessary miniaturization and reduction in power consumption of the implantable system.

However, as the size of the implant gets reduced, the exact location of the implant gets difficult to ascertain, hence the positional placement of the external proximity communicator (used for powering and data communication above the skin) becomes challenging. The aforementioned alignment issue gets further complicated as both devices (i.e. proximity communicator mostly and to a lesser extend the implanted device) tend to migrate from their initial sites. In addition, the implanted sensor, as its footprint decreases, it also has the tendency to rotate both perpendicularly as well as along its long axis. To this end, the positional detection of the implant becomes crucial to the success of collecting real-time sensor data from the implant, as the powering and output data communication components on the implantable device need to be properly aligned with both the power source and the data communications receiver located in the external unit. This necessitates a methodology to detect the location of and align with the biomedical implant which will allow for facile integration to the current implantable platform. Furthermore, such positional detection scheme should ideally do not add extra weight, size or increased power consumption to the overall system.

Positional sensing of implantable medical devices has been achieved in varying forms via employing externally induced electromagnetic fields from the external communicator through the users' skin. In one approach, U.S. Pat. No. 8,473,066 B2 describes a scheme where the induced electromagnetic field used to power the implanted device is reflected back to the external charger/reader, as shown in FIG. 1A. Here, the induced electromagnetic field is reflected back by the coil used to inductively power the implanted biomedical device and by the use of multiple staggered coils on the external unit, x- and −y orientation can be determined depending upon the magnitude of the reflected signal. In another methodology, US Patent Application US 2010/0010338 A1 describes an invention wherein the location of an implantable biomedical device is determined using an accelerometer embedded in the implant for the determination of x- and y-coordinates, as illustrated in FIG. 1B. Z-coordinates are obtained by the use of an external magnet located in the external communicator in conjunction with a magnetic sensor embedded in the implanted device. Here, it is noteworthy to mention that the previously disclosed invention employs the use of an integrated magnetic sensing circuit and accelerometer embedded in the implanted biomedical platform. The addition of these circuits inherently results in both a larger implantable device footprint as well as an increase in the overall power consumption of the device. The present invention addresses this issue in that it integrates static magnetic materials directly in the implant which do not draw any power from the implanted power source.

### SUMMARY OF THE INVENTION

The present invention provides a methodology for pinpointing the location of an implantable biomedical sensing device. In one embodiment, this is carried out by integrating miniaturized magnets, or materials with magnetic properties into the implantable bio-sensing chip to detect the position of the implant by sensing the induced magnetic field wirelessly by an external proximity communication unit. Presented here are various configurations in which magnetic positional detection can be carried out. The positional information collected from these detection motifs can be used to provide feedback to the user about alignment status as well as activate a self-alignment methodology.

In one embodiment, the implantable biosensor platform may include bar code type 1-D, and/or QR code type and/or AR code type 2-D patterns (also any other types of bar codes may be used, such as 3-D and image codes etc.) that use reflecting coatings comprised of metals, dielectric mirrors and/or phosphors sensitive to the lighting source that powers the solar cells. The coatings may be located on one of the sub-chips. The imaging may be done by a CCD or MOS camera in the external control unit. Once the implanted biosensor is located and aligned, locking of the position is achieved by energizing the electromagnetic coils located in the external control unit.

In yet another embodiment, magnetic sensors may be used on the implantable biosensor platform and located on the surface of one of the sub-chips. The magnetic sensors may map the magnetic field produced by the electromagnetic coils in the external unit.

### BRIEF DESCRIPTION OF THE FIGURES

The foregoing and other features and advantages of the present invention will be more fully understood from the following detailed description of illustrative embodiments, taken in conjunction with the accompanying drawings in which like elements are numbered alike in the several Figures:

FIG. 1A illustrates the detection of a biomedical implant based on reflectance of an induced magnetic field, in accordance with the prior art.

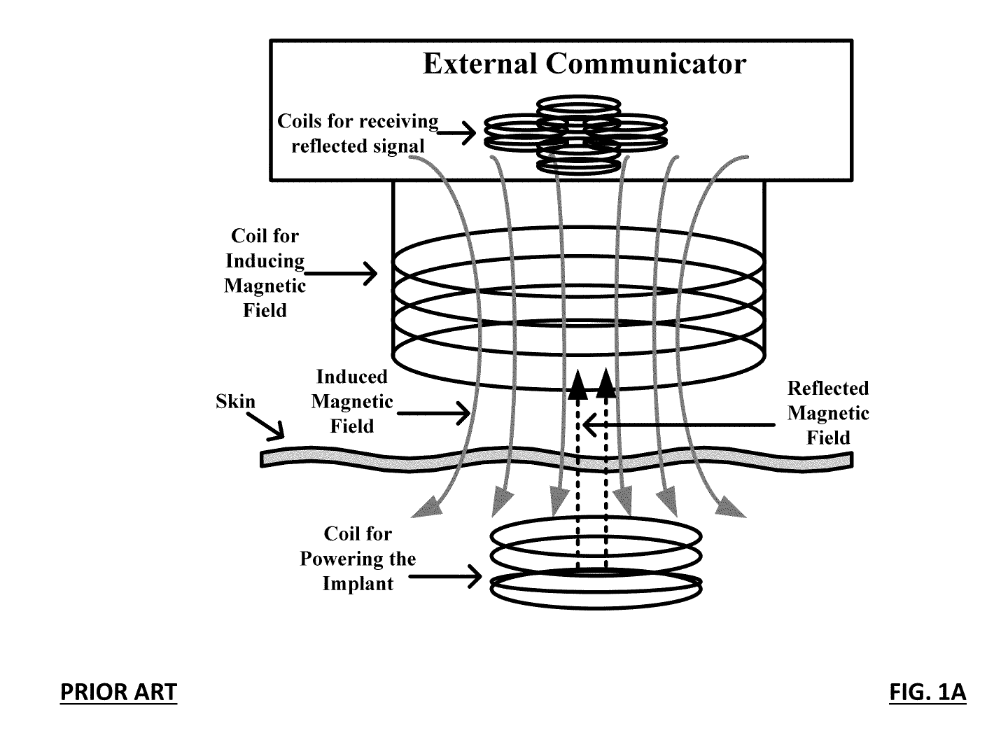

FIG. 1B illustrates the detection of a biomedical implant based on x-, y- and z-axis orientation using an accelerometer (x- and y-) as well as an external magnet and internal magnetic sensor (z-axis), in accordance with the prior art.

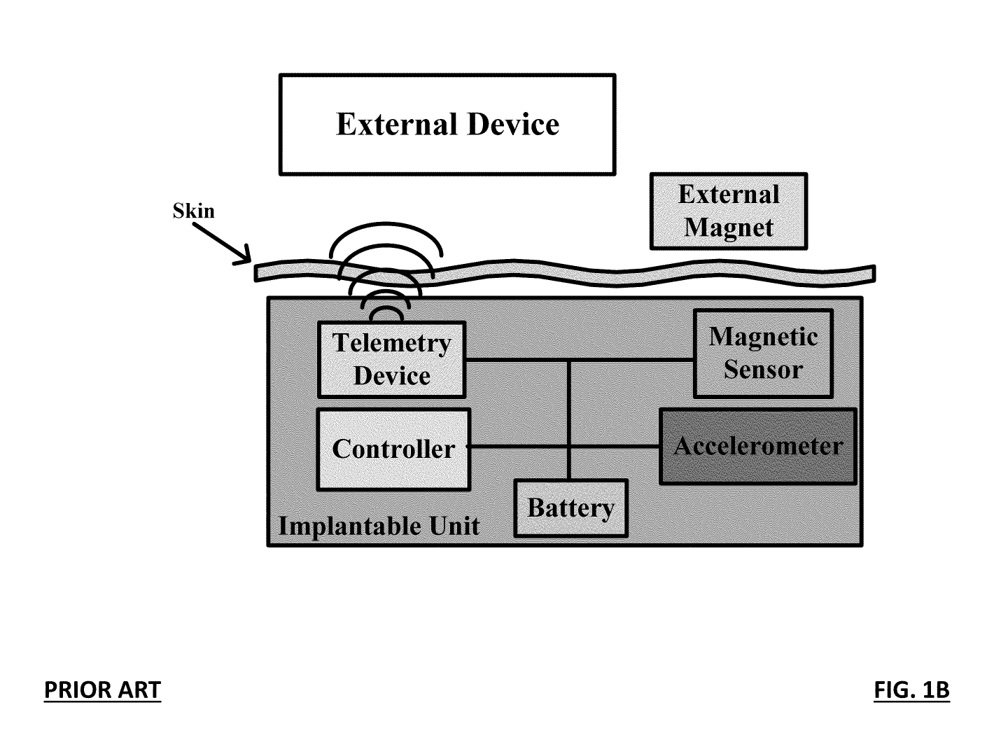

FIG. 2 illustrates a magnetic self-alignment scheme, in accordance with one embodiment of the invention, which employs two miniature magnets on both the proximity communicator as well as the implantable unit.

FIG. 3A is a cross-sectional schematic representation of the magnetic detection scheme of FIG. 2.

FIG. 3B is a cross-sectional schematic representation of a magnetic detection scheme, in accordance with another embodiment wherein the position of the implant is detected by magnetic sensors, and when found, an electromagnet in the proximity communicator is energized, locking the two together.

FIG. 3C is a cross-sectional schematic representation of a magnetic detection scheme in accordance with yet another embodiment which utilizes metal detection in the proximity communicator, where audio and/or visual feedback is relayed back to the user to improve alignment between the communicator and implant.

FIG. 4 is another embodiment of a magnetic self-alignment scheme which utilizes a freely-floating integrated device consisting of the powering component, data communication receiver and magnet, wherein the implantable platform is equipped with the corresponding powering receiver, data communication component as well as a magnet. The freely-floating magnetic device in the proximity communicator is then able to migrate to the implantable platform via magnetic attraction.

FIG. 5A illustrates a magnetic positioning methodology in accordance with one embodiment, which utilizes an x-y stepper motor to align the powering and data receiving components on the proximity communicator together with the corresponding power receiver and data transmitter components on the implantable sensor platform. The hall sensors or other magnetic detection mechanism receives information about the location of the implantable unit based on the magnitude of the magnetic field emitted from the implantable platform.

FIG. 5B illustrates a methodology of employing electromagnets in the proximity communicator which are energized based on the alignment information provided by the magnetic sensing circuits, in accordance with another embodiment.

FIG. 6A illustrates a magnetic positional detection scheme, in accordance with another embodiment, wherein the proximity communicator is outfitted with an array of devices consisting of magnetic sensors/powering transmitter/data receiver, such that when brought into close contact with the implant, the proximity communicator activates the close device to the implantable sensor platform based on the magnitude of the magnetic field emitted from the implant.

FIG. 6B illustrates a magnetic positional detection scheme similar to that of FIG. 6A which includes electromagnets and indicators (audio/visual), where the electromagnets get energized when aligned thereby locking into place, in accordance with another embodiment.

FIG. 7 illustrates a cross-section of an implantable biosensor platform, in accordance with another embodiment, having bar code type 1-D and AR code type 2-D patterns using reflecting coatings comprising of metals, dielectric mirrors, phosphors sensitive to the lighting source that powers the solar cells. Coatings are located on one of the sub-chips, where the imaging is done by a CCD or MOS camera in the external control unit. Once the implanted biosensor is located and aligned, locking of the position is achieved by energizing the electromagnetic coils located in the external control unit.

FIG. 8 is a cross-section of an implantable biosensor platform showing magnetic sensors on the implantable biosensor platform on the surface of one of the sub-chips, in accordance with one embodiment of the invention. The magnetic sensors map the magnetic field produced by the electromagnetic coils in the external unit.

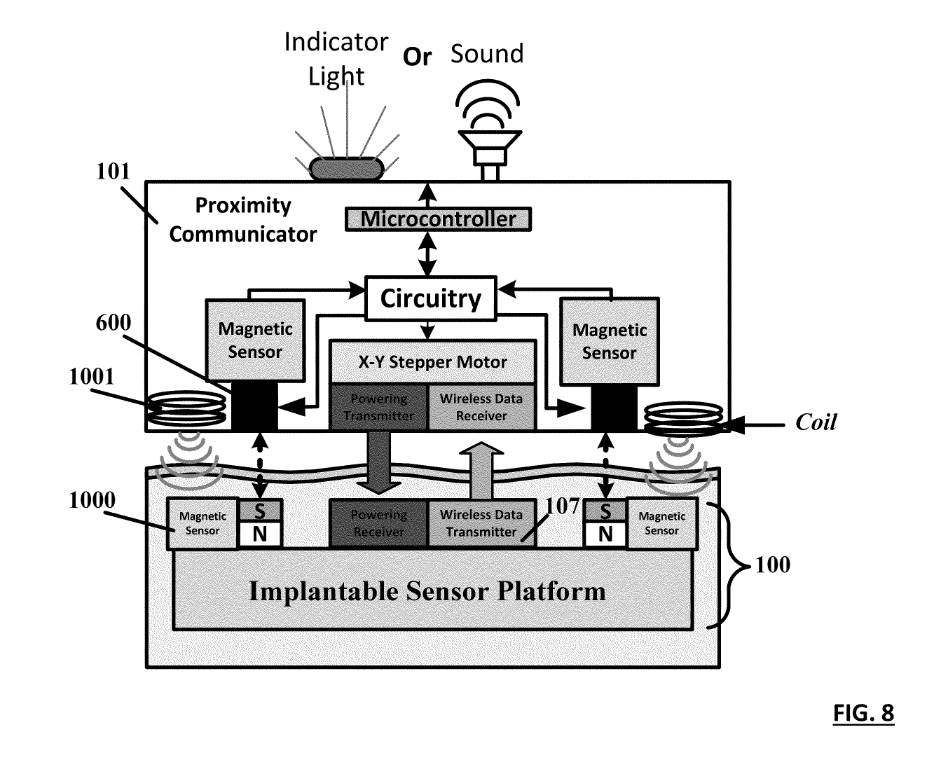

FIG. 9A shows one embodiment of a current implantable prototype and a glass carrier substrate integrated with two miniature magnets.

FIG. 9B shows one embodiment of a magnetic locking scheme with a current implantable prototype and a glass substrate mounted with magnets.

FIG. 10A shows an implantable prototype sensor (bottom), in accordance with one embodiment of the invention, integrated with a miniature magnet, while the proximity communicator (top) is equipped with a magnetic sensing circuit.

FIG. 10B shows a magnetic sensing scheme in accordance with one embodiment of invention through a rat skin, where the implantable prototype sensor is aligned to the magnetic sensing circuit of the proximity communicator of FIG. 10A, activating a secondary LED indicator to signal successful alignment.

### DETAILED DESCRIPTION OF THE INVENTION

The present invention provides a methodology to pinpoint the location of a miniaturized implantable biomedical device by integrating the implant with miniature magnets or materials with magnetic properties (such as nano-sized iron particles or other polarized nanomaterials). Once integrated into the implanted platform, the magnetic field generated through the patient's skin can then detected by the proximity communicator unit located externally in a variety of techniques. These techniques can be partitioned into two distinct methodologies, namely: (i) “self-alignment” methodology, which automatically aligns the powering and data communication components on the implantable platform together with the powering and data receiving components on the external communicator unit when both are in close proximity to each other, and (ii) “User-controlled” alignment, wherein the alignment/misalignment of the implantable unit relative to the proximity communicator is relayed to the user via either audio or visual means, prompting the user to make the necessary alignment adjustments. Here, it is noted that addition to outfitting the implant with miniature sized magnets, electromagnetic device recognition can also be carried out by employing metal detection schemes, utilizing the gold frame used to hermetically seal the chip from the surrounding biological fluid. This gold framing methodology has been previously disclosed by our group in a previous patent.

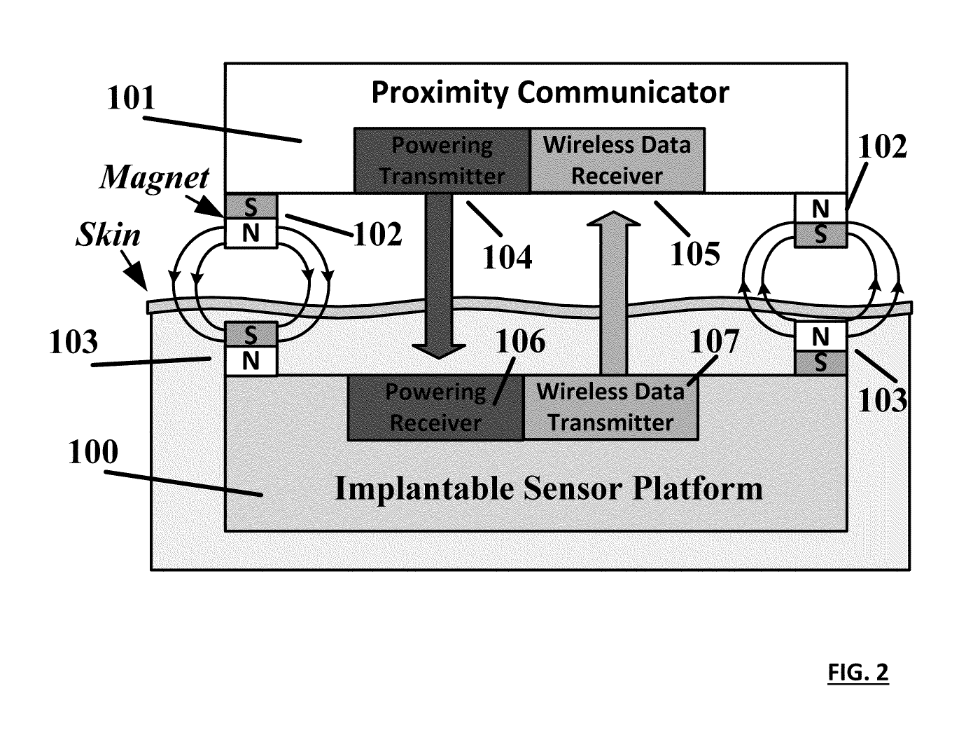

In its simplest form, FIG. 2 displays a cross-sectional schematic representation of the implantable unit 100 and external proximity communicator 101 which are both equipped with two miniature magnets, where the two magnets 102 on the proximity communicator are separated at an equal distance relative to the magnets 103 on the implantable unit. Similarly, the powering transmitter component 104 and data receiving component 105 on the proximity communicator are separated at an equal distance from each other relative to the powering receiver component 106 and data communication transmitter component 107 on the proximity communicator.

It is noted that the magnets on the implantable unit 100 and proximity communicator 101 are placed such that opposite poles are facing each other. In doing so, when the proximity communicator 101 is brought in the vicinity of the implantable unit 100, the magnetic field 108 of the magnets locks the implant 100 to the proximity communicator 101, thereby aligning the powering 106/data communication 107 components of the implantable unit 100 with that of the proximity communicator 101 and preventing the implantable device 100 from rotating and/or migrating from its current location. Henceforth, the aforementioned alignment/positioning approach alleviates the need for the user to perform routine positional adjustments of the proximity communicator 101 as misalignment occurs.

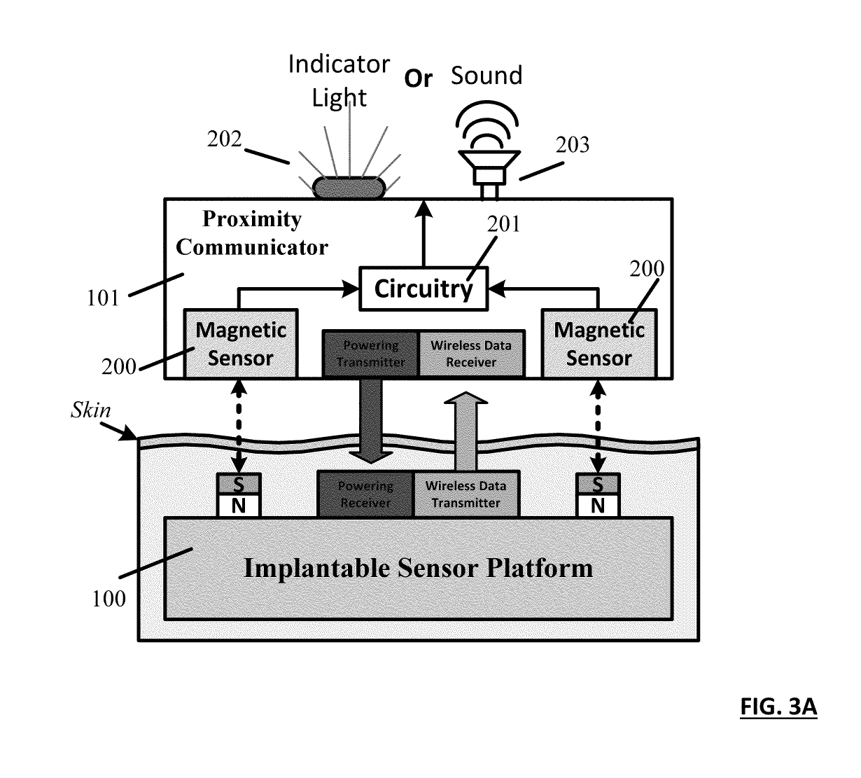

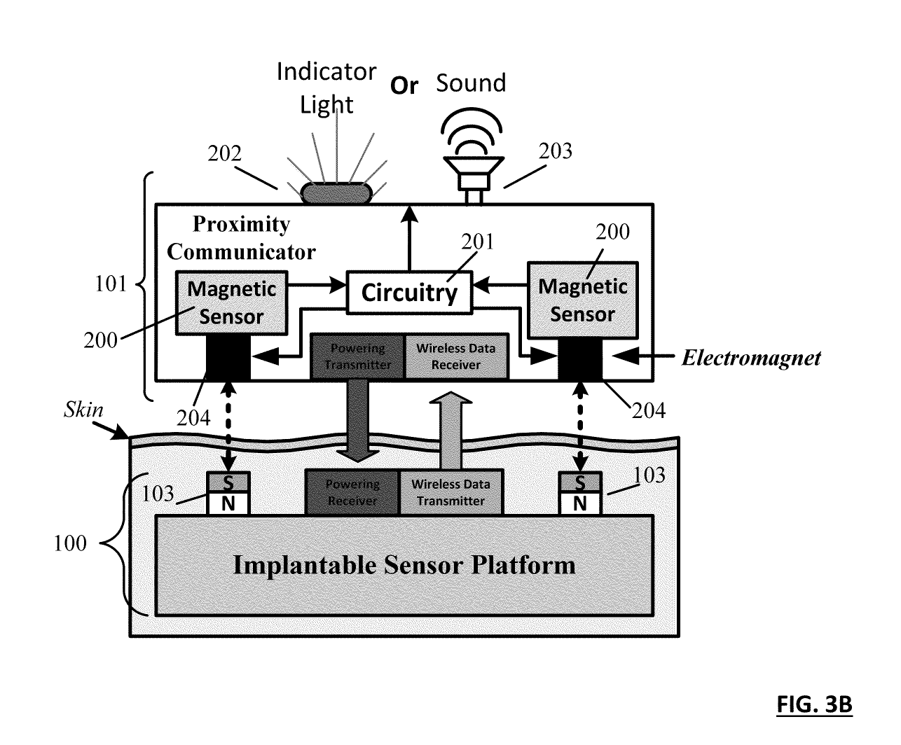

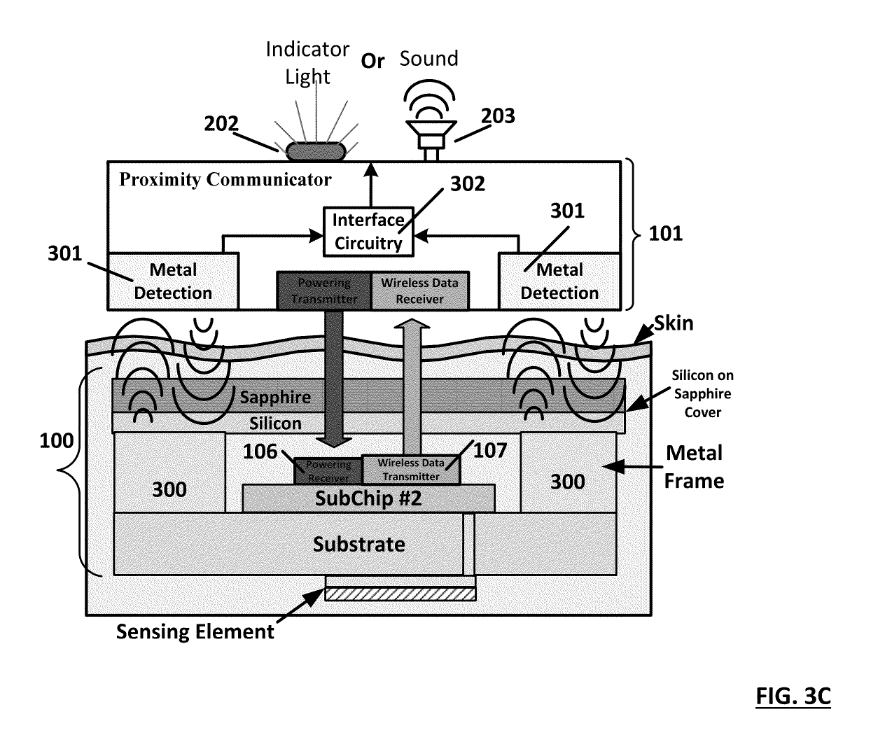

In another approach of the present invention, magnetic detection of the implantable sensor chip 100 is carried out by outfitting the proximity communicator 101 with magnetic sensors 200 such as hall sensors (also known as hall-effect devices) along with their corresponding circuitry 201 which align with the magnets 103 located on the implantable unit 100. This methodology is shown in FIG. 3A. As the proximity communicator 101 is brought in the vicinity of the implantable device 100 the magnetic field 108 is sensed by the proximity communicator, and based on the magnetic field strength received, the magnet sensors 200 and circuits 201 located in external unit activate an indicator light 202 or sound 203 which indicates alignment. Based on this feedback, the user then performs the required adjustment of the proximity communicator 101 to achieve alignment. A variation on this positional detection scheme is shown in FIG. 3B and involves the use of electromagnets 204, rather than discrete magnets 103, along with magnetic sensing circuits 201 in the proximity communicator. In this method, once the magnetic sensing circuits 201 have indicated alignment, the electromagnets 204 are then activated which locks the proximity communicator 101 to the implant 100.

In another approach, rather than integrating discrete magnetic components onto the implantable platform, this scheme involves the location of the metal frame 300 used for hermetically-sealed operation (FIG. 3C). Here, the proximity communicator 101 is outfitted with metal detection components 301 and associated circuitry 302, which provide feedback to the user via audio 203 or visual 202 means about if the powering 106/communication 107 components on the implantable device 100 is aligned properly with those on the proximity communicator 101.

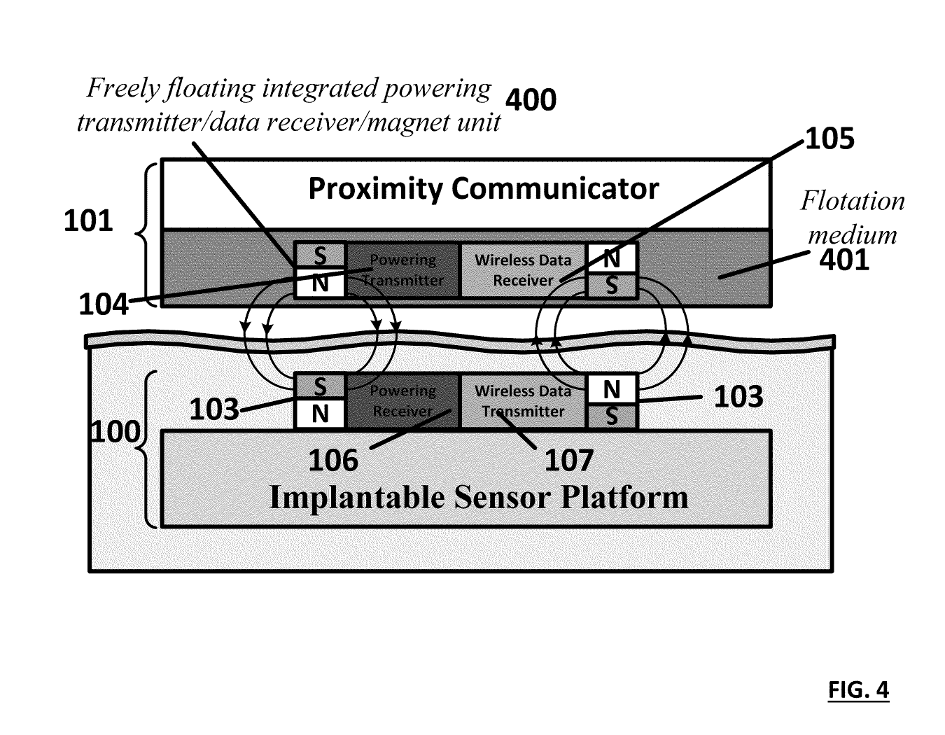

FIG. 4 displays a cross-sectional schematic representation of an embodiment of the “self-alignment” scheme of magnetic positioning. In this approach, the powering component 104, data communication receiver 105 and magnet are all integrated into a single device 400 which is floating in an oil 401 or other medium which allows the device to move freely in the −x and −y directions.

On the implantable unit 100, the powering receiver component 106, data communication component 107 and magnet 103 are all separated at equal distances relative to the freely-floating integrated device located in the proximity communicator 101. When the external unit 101 is brought in the vicinity of the implantable device 100, the magnetic attraction between the magnet 102 located on the proximity communicator 101 and the implantable chip 103 (with opposite poles facing each other) allows the integrated powering/data receiver/magnetic component on the proximity communicator 101 to find and align with the implantable sensor chip 100.

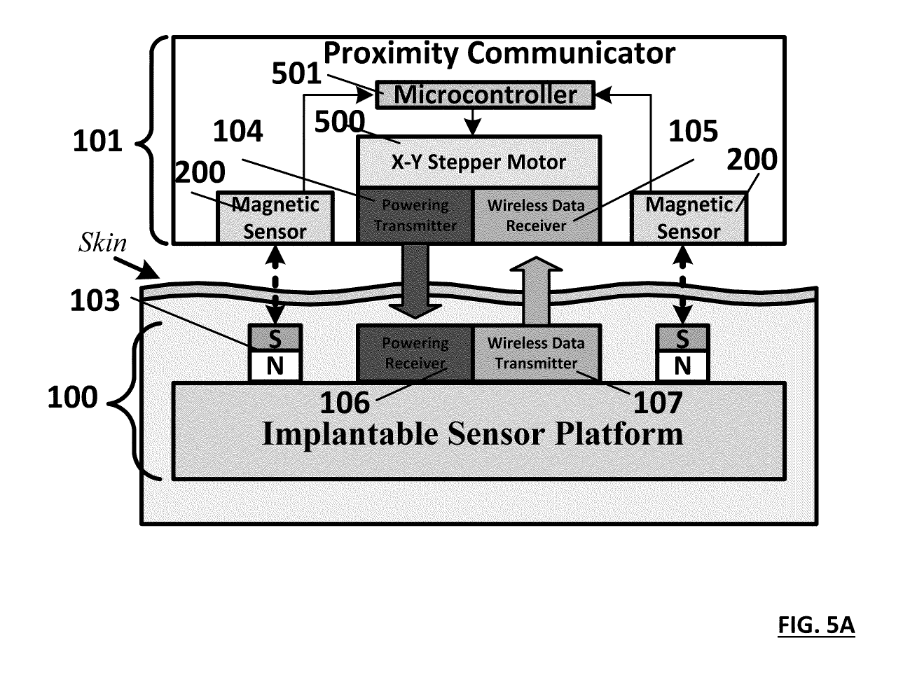

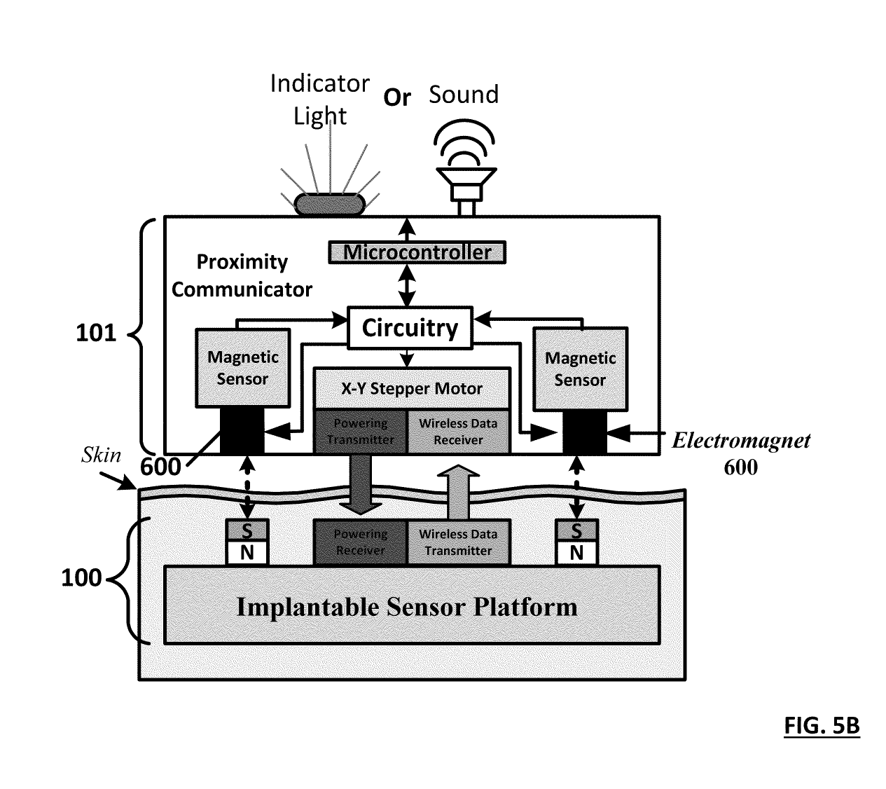

In a similar approach, an x-y stepper motor 500 is employed to align the powering 104 and data receiving components 105 on the proximity communicator 101 together with the corresponding power receiver 106 and data transmitter components 107 on the implantable sensor platform. FIG. 5A illustrates this scheme, where a set of magnetic sensors 200, such as hall sensors or other magnetic detection mechanism, receives information about the location of the implantable unit 100 based on the magnitude of the magnetic field emitted from the implantable platform 100. Based on this positional information, the microcontroller 501 drives the x-y stepper motor 501 thereby repositioning the powering communication components in the proximity communicator 101 to the correct location above the implantable unit 100. A similar variation of this scheme can be realized by integrating the proximity communicator 101 with an electromagnet 600, wherein the electromagnet is energized upon successful alignment via the magnetic sensor 200 and locks the communicator 101 to the implant 100, as shown in FIG. 5B. Here, light 202 and/or sound 203 components are activated once alignment is achieved.

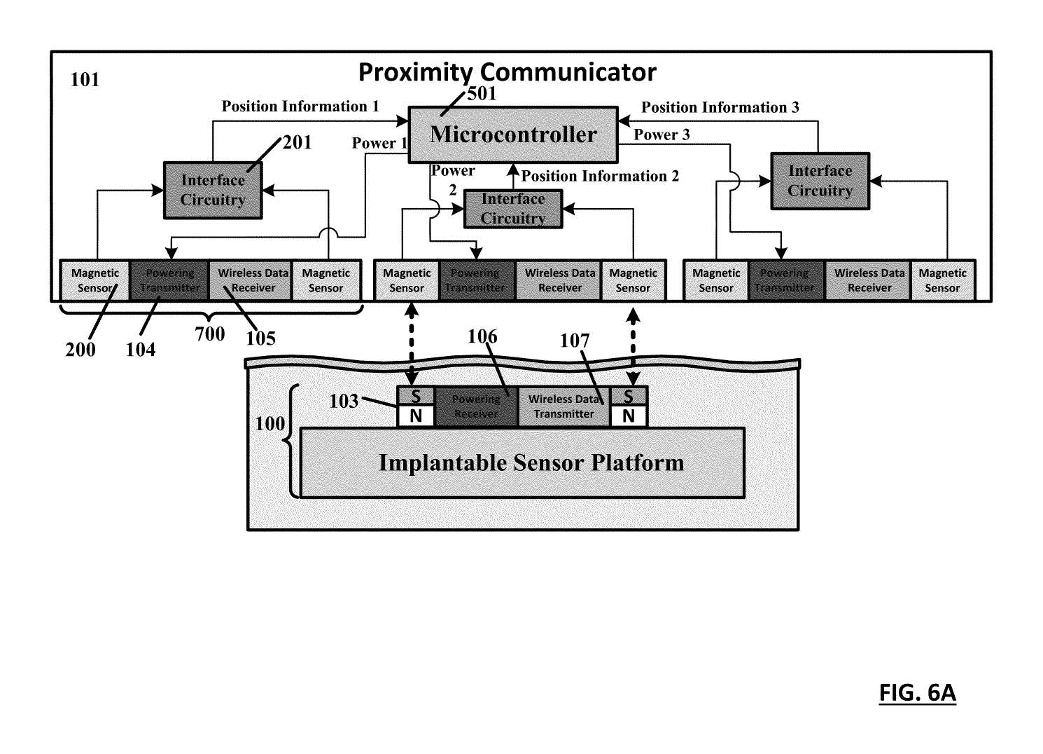

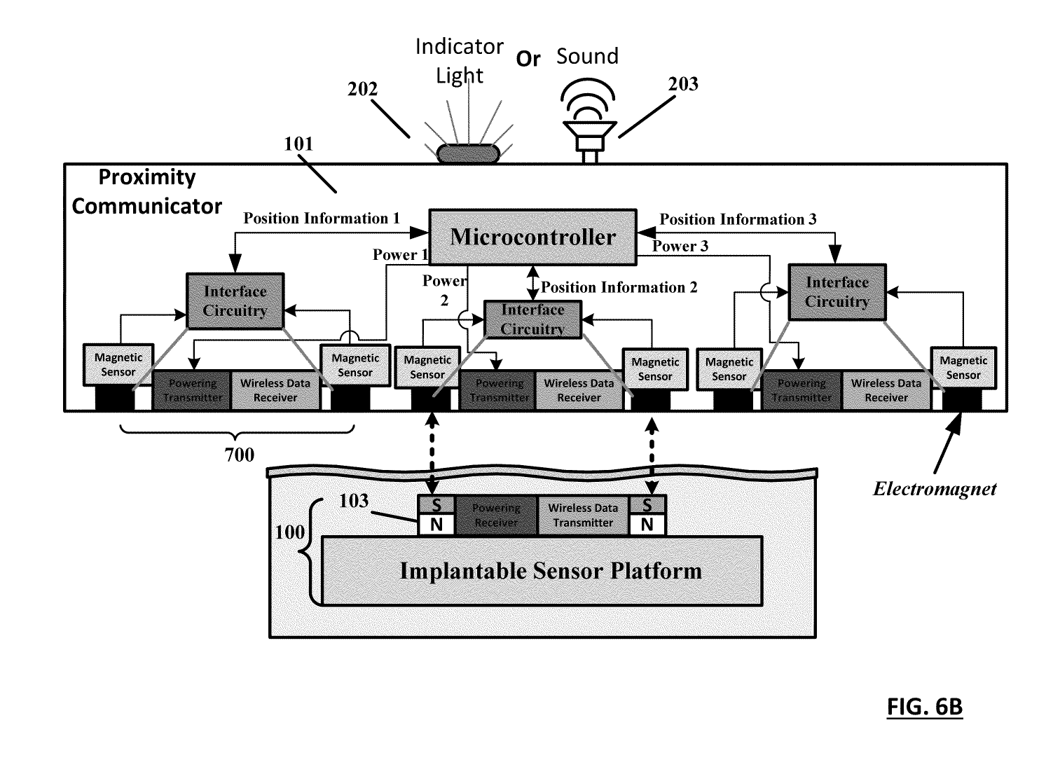

In yet another embodiment of the present invention, an array of devices 700 consisting of magnetic sensors 200, powering transmitter 104 and data communication receiver 105 (each of which align with the magnets 103, powering receiver component 106 and data transmitter 107 on the implantable unit 100) are positioned at various locations in the external proximity communicator 101, as shown in FIG. 6A. Additionally, each device interfaces with the microcontroller 501 and associated interface circuitry 201. As the user brings the proximity communicator 101 within the vicinity of the implantable unit 100, the magnetic positioning information is relayed to the microcontroller 501 which determines which magnetic sensors/powering transmitter/data communication receiver device 700 is closest with the corresponding magnets 103, powering receiver component 106 and data transmitter 107 on the implantable unit 100. Once the closest has been determined, the microcontroller 601 activates the aforementioned device 700 which then powers and communicates with the implantable unit 100.

By outfitting the proximity communicator 101 in this fashion, the implant will always be receiving input power from one of the power/communication devices 700 in the external unit even if it migrates from the initial implantation site. Here, it is noted that this proximity communicator architecture can also be outfitted with electromagnets 600 and an audio 203/visual 202 feedback system as illustrated in FIG. 6B. Here, when the magnets 103 on the implantable unit 100 get aligned with one of the powering/communication receiver units 700 in the proximity communicator 101, the electromagnets 600 in the proximity communicator 101 get energized, thereby locking into place.

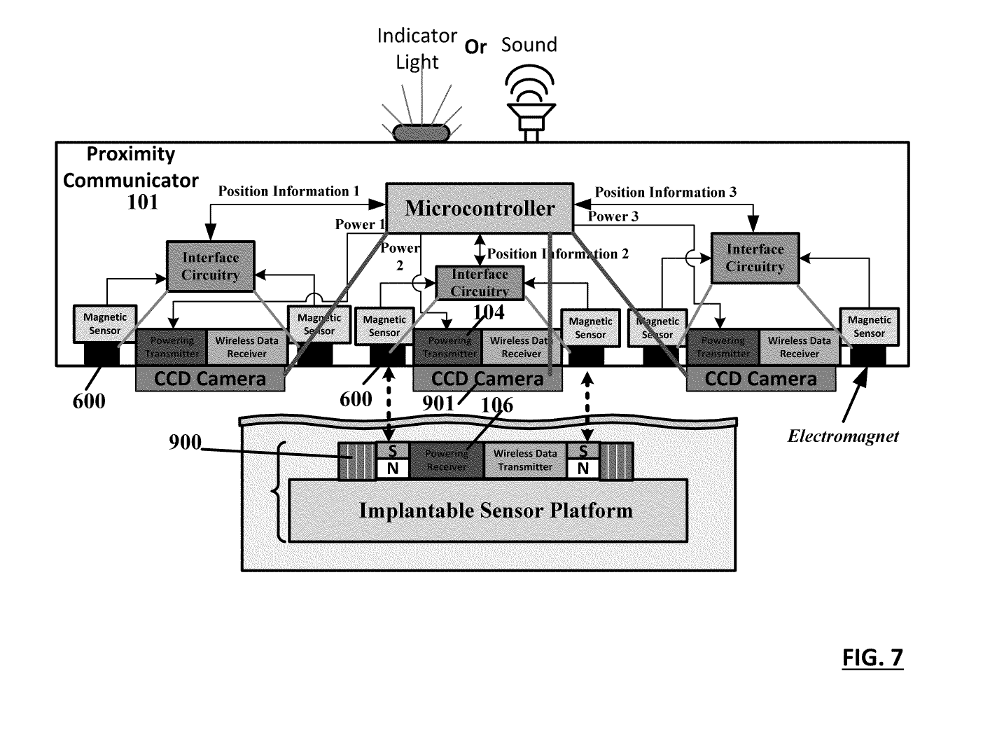

In yet another approach, the implantable biosensor platform 100 having bar code 900 type 1-D and AR code type 2-D patterns using reflecting coatings comprising of metals, dielectric mirrors, phosphors sensitive to the lighting source 104 that powers the solar cells 106 is shown in FIG. 7. Coatings are located on one of the sub-chips. The imaging is done by a CCD or MOS camera 901 in the external control unit 101. Once the implanted biosensor 100 is located and aligned, locking of the position is achieved by energizing the electromagnets 600 located in the external control unit 101.

In yet another scheme, magnetic sensors 1000 are located on the implantable biosensor platform 100 on the surface of one of the sub-chips. The magnetic sensors 1001 map the magnetic field produced by the electromagnetic coils 1001 in the external unit which are then relayed back to the proximity communicator 101 via the optical transmitter 107. Here, electromagnets 600 in the proximity communicator 101 align and lock with the implantable unit 100.

A series of experiments have been carried out to illustrate the implementation of this technology, however it is noted that the present invention includes, but is not limited to the following examples.

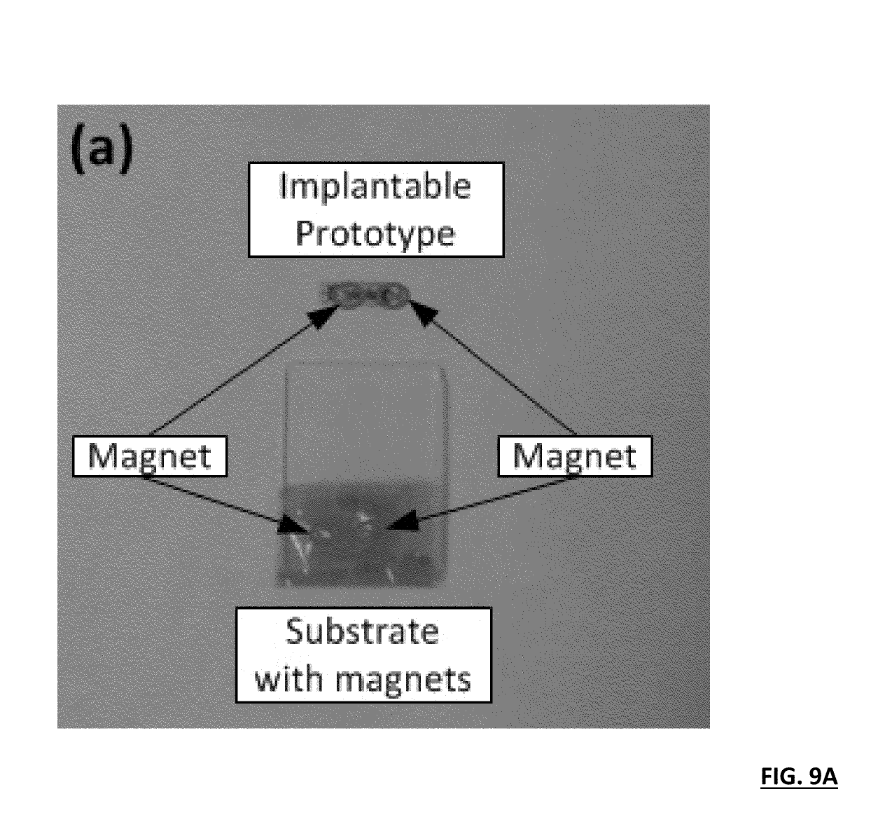

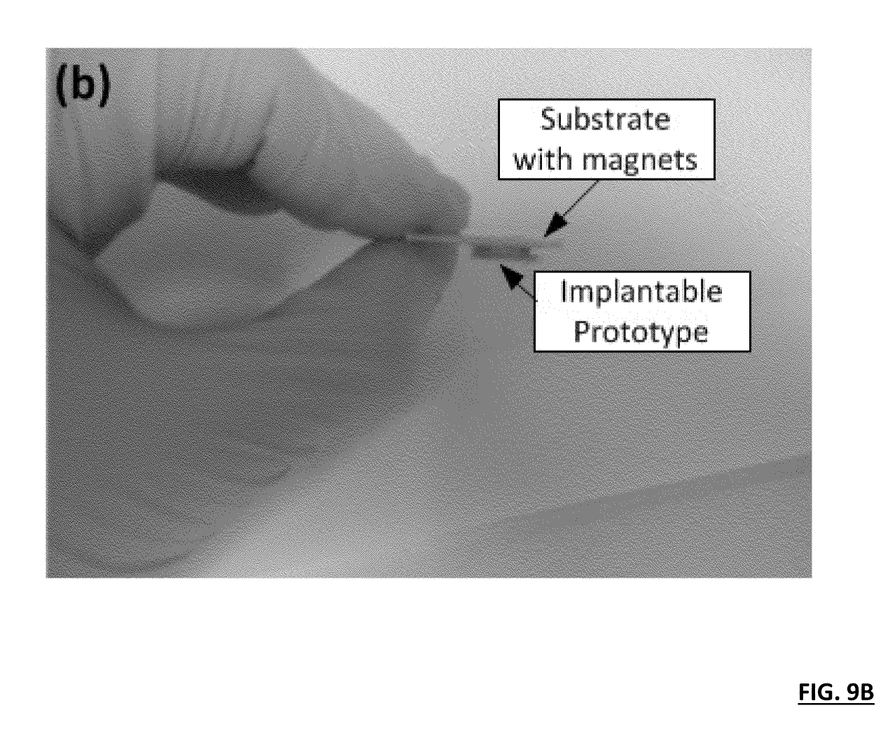

The magnetic locking scheme of FIG. 2 has been successfully demonstrated. Here, we have integrated our current prototype with miniature magnets at opposite ends of the device, as shown in FIG. 9A. Magnets with opposite poles facing outward at an equal separation distance were then fastened to a carrier substrate which is intended to represent the proximity communicator. When brought in close contact to the implantable prototype, the magnetic attraction between the substrate and device locks each other together. This is illustrated in FIG. 9B, where the implant prototype is fastened to the carrier substrate solely by magnetic attraction.

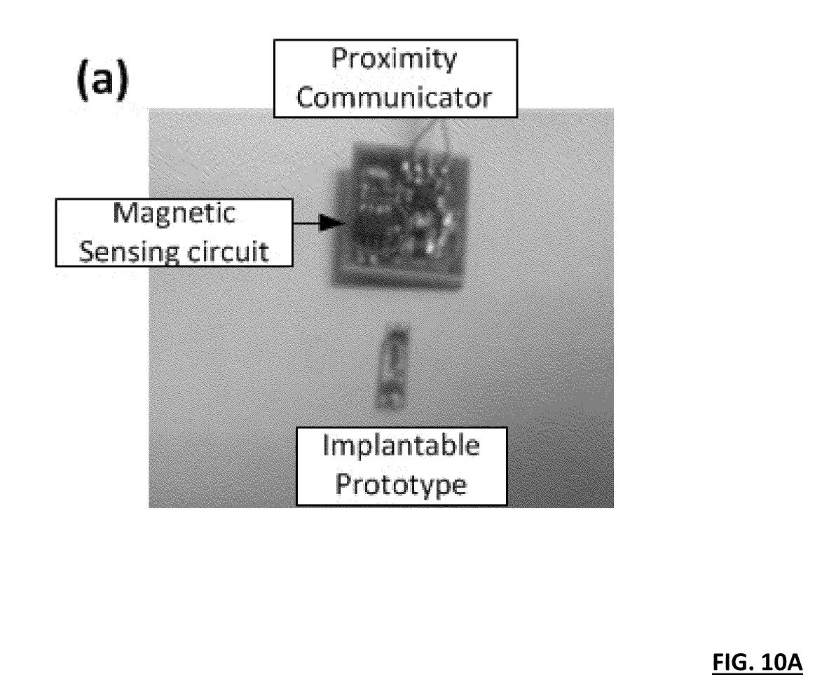

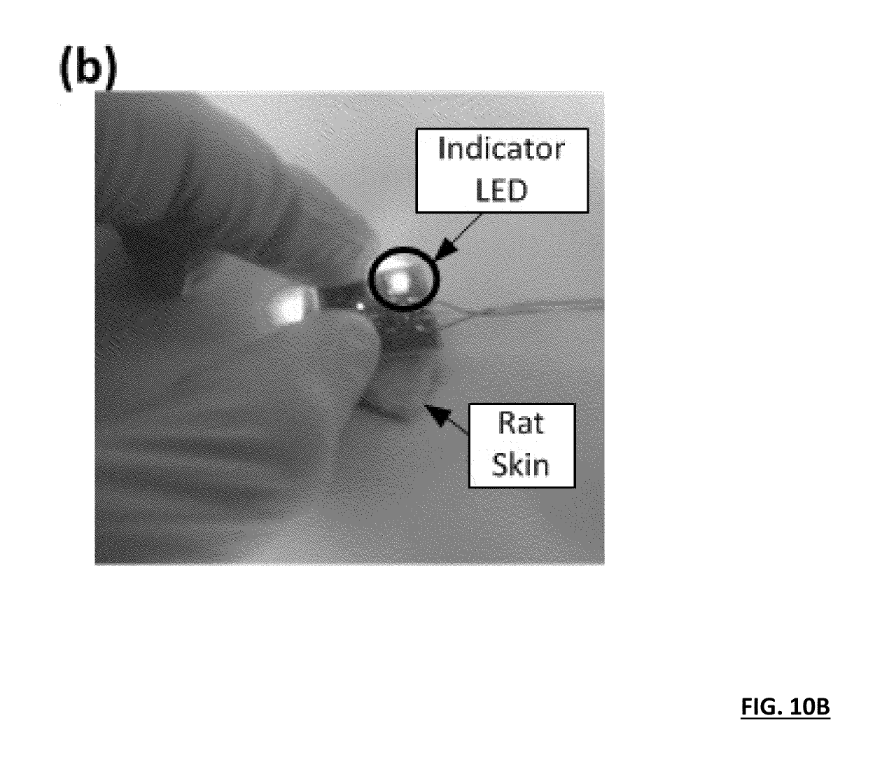

In addition, we have implemented the approach of FIG. 3 by integrating a prototype proximity communicator (complete with powering and communication components) with a magnetic sensing device as well as an indicator LED, shown in FIG. 10A and FIG. 10B. Here, a piece of rat skin was inserted between the implantable prototype and the proximity communicator. As the proximity communicator was brought in the vicinity of the magnet on the integrated prototype device, the magnetic field was sensing through the rat skin and the red LED turned on, indicating successful alignment. In this figure, there are two sources of red light; the large background red light is due to the powering LED located on the bottom of the proximity communicator, and the top red light is the indicator LED which indicates alignment (circled in RED).

In accordance with one embodiment of the invention, a method of detecting and locating the position of an implanted biosensor platform which is in communication with an external control unit, providing levels of designated analytes in subcutaneous tissue, is provided, wherein the biosensor platform is optically powered by solar cells and is in wireless communication with the external control unit via optical communication using a photodetector(s) serving as a receiver which receives coded instructions and transmitting analyte levels and other conditions such as power level received by solar cells using a dedicated optical transmitter. The external unit may include a camera which enables the operator to position it over the implanted biosensor platform, the camera images the biosensor platform using light source in the external unit that powers the solar cells or photovoltaic devices located on one of the sub-chips in the implanted biosensor platform, the biosensor platform may include sensor electrodes dedicated for measuring designated analytes such as glucose, lactate, oxygen and pH, and wherein the biosensor platform is coated with biocompatible coatings and is sealed against body fluids on all sides with the exception of the sensor electrodes. Moreover, the biosensor platform may include one or more Si integrated circuit sub-chips configured to perform signal processing functions needed in the measurement of analyte levels, surface of one or more of the sub-chips may include patterned magnetic regions serving as source of magnetic field, wherein the magnetic regions may include a high strength magnetic material selected from a list which includes samarium, iron, ferrite, and/or samaraium boron garnet, and wherein the magnetic field is detected and measured by two or more magnetic sensors located in the external control unit, wherein the magnetic sensors in cooperation with the microprocessor unit in the external control unit produce a 3-dimensional map of the location of the implanted biosensor platform relative to the location of the external control unit, the external control unit including an x-y stage which can be moved with respect to its frame relative to the location of the implanted biosensor platform so that a desired optimal magnetic field distribution is obtained.

In accordance with another embodiment of the invention, a method of detecting, locating the position, and aligning an implanted biosensor platform relative to an external control unit, providing with reproducible measurement of levels of designated analytes in the subcutaneous tissue in which the biosensor is implanted, is provided, wherein the external control unit comprising of one or more electromagnet coils which upon energizing by passing current of desired magnitude enables the alignment of the implanted biosensor platform, wherein the biosensor platform is optically powered by solar cells and is in communication wirelessly with the said external control unit via optical communication using photodetector serving as receiver which receives coded instructions and transmitting analyte levels and other conditions such as power level received by solar cells using a dedicated optical transmitter, the solar cells receives light power from at least one source located in the said external control unit, the external unit comprises of a camera which enables the operator to position it over the implanted biosensor platform, the biosensor platform comprises of sensor electrodes dedicated for measuring designated analytes such as glucose, lactate, oxygen and pH, and wherein the biosensor platform is coated with biocompatible coatings and is sealed against body fluids on all sides with the exception of the sensor electrodes, the biosensor platform comprises of one or more Si integrated circuit sub-chips performing signal processing functions needed in the measurement of analyte levels, surface of one or more said sub-chips comprising of patterned magnetic regions serving as source of magnetic field, the magnetic regions is comprised of high strength magnetic material selected from a list samarium, iron, ferrite and/or samaraium boron garnet, wherein the magnetic field is detected and measured by two or more magnetic sensors located in the external control unit, the magnetic sensors in cooperation with the microprocessor unit in the external control unit produces a 3-dimensional map of the location of said implanted biosensor platform relative to the location of the external control unit, the surface of the sub-chips in implanted biosensor platform comprising of more than one magnetic field sensors located such that the magnetic sensor minimally receives the magnetic field of the magnetic regions, the magnetic field sensors located on sub-chips measure the magnetic field of the electromagnetic coils housed in the external control unit, and transmit magnetic field magnitude and their distribution information using the optical transmitter of the implanted unit, the magnetic field distribution of the electromagnetic coils is received by the photodetector of the external control unit, the magnetic field data of the electromagnetic coils is processed by the microprocessor unit in the external control unit, and produces a 3-dimensional map of the location of the implanted biosensor platform magnetic sensors relative to the location of the electromagnetic coils, the external control unit comprising of an x-y stage with provision for tilting along two axes, the x-y stage can be moved with respect to external control unit frame relative to the location of the implanted biosensor platform so that a desired optimal alignment is obtained after processing the two sets of magnetic field distribution maps, the implanted biosensor platform is locked with the help of magnetic field created by electromagnetic coils for repeated analyte measurements.

In accordance with still yet another embodiment of the invention, a method of detecting, locating the position, and aligning and locking in position an implanted biosensor platform relative to an external control unit, providing reproducible measurements of levels of designated analytes in the subcutaneous tissue in which the said biosensor is implanted is provided, wherein detection and location of position of the implanted biosensor platform is done by optical pattern recognition methods using a camera and imaging device and light sources housed in the external control unit, and optical contrast producing patterns located on the surface of sub-chips in the implanted biosensor platform, wherein the optical contrast producing patterns are coded with 1-dimensional bar code, 3-dimensional, QR codes and/or 2-dimensional AR code, the coded bar code patterns are implemented using reflective metal patterns, semiconducting patterns or phosphor material patterns which are generating reflections at the wavelength of powering source that powers the solar cells, wherein the imaging device may include components from a list of devices such as a 2-dimensional charge coupled device (CCD), a 2-D metal-oxide semiconductor (MOS) imaging device, wherein the biosensor platform is optically powered by solar cells and is in communication wirelessly with the external control unit via optical communication using photodetector serving as receiver which receives coded instructions and transmitting analyte levels and other conditions such as power level received by solar cells using a dedicated optical transmitter, the implanted biosensor platform, wherein precision alignment and locking of the external control unit is done by electromagnetic coils, x-y stage with 2-axis tilting and rotation features, located in the external control unit, the electromagnetic coils function in cooperation with magnetic field sensors and magnetic regions located on the sub-chip surface of the implanted unit, the external control unit comprising of one or more electromagnet coils which upon energizing by passing current of desired magnitude enables the alignment of the implanted biosensor platform, the optical pattern detection and alignment and magnetic locking is done in cooperation with the external control unit microprocessor and its algorithms.

In accordance with still yet another embodiment of the invention, a device as described herein is provided where the powering component, data communication receiver and/or magnets may be integrated into a single device which is floating in an oil or other medium allowing the device to move freely in the −x and −y directions, wherein the integrated powering/data receiver/magnetic component in the external unit finds and aligns with the powering receiver and data communication component on the implantable via magnetic attraction. Additionally, the powering component, data communication receiver and/or magnets may be integrated into a single device which is floating in an oil or other medium allowing the device to move freely in the −x and −y directions and the integrated powering/data receiver/magnetic component in the external unit may be configured to find and align with the powering receiver. Additionally, the powering component, data communication receiver and/or magnets are all integrated into a single device which is floating in an oil or other medium allowing the device to move freely in the −x and −y directions, wherein the integrated powering/data receiver/magnetic component in the external unit may be configured to find and align with the powering receiver.

In accordance with still yet another embodiment of the invention, a device as disclose herein, where an x-y stepper motor and associated circuitry is integrated into the external proximity communicator; the x-y stepper motor is driven by positional information provided by the magnetic sensors located in the external unit; the x-y stepper motor is driven by a microcontroller and interface circuitry based on positional information provided by the magnetic sensors; the x-y-stepper motor aligns the powering and data receiver components on the proximity communicator with the power receiving and data communication components on the implantable unit.

In accordance with still yet another embodiment of the invention, a device as disclose herein, where an x-y stepper motor and associated circuitry is integrated into the external proximity communicator; the x-y stepper motor is driven by positional information provided by the magnetic sensors located in the external unit; the x-y stepper motor is driven by a microcontroller and interface circuitry based on positional information provided by the magnetic sensors; the x-y-stepper motor aligns the powering and data receiver components on the proximity communicator with the power receiving and data communication components on the implantable unit.

In accordance with still yet another embodiment of the invention, a device as disclose herein, where an x-y stepper motor and associated circuitry is integrated into the external proximity communicator; the x-y stepper motor is driven by positional information provided by the magnetic sensors located in the external unit; the x-y stepper motor is driven by a microcontroller and interface circuitry based on positional information provided by the magnetic sensors; the x-y-stepper motor aligns the powering and data receiver components on the proximity communicator with the power receiving and data communication components on the implantable unit.

In accordance with still yet another embodiment of the invention, a method to detect the special location of an implantable biosensor is provided and includes, miniature magnets or materials with magnetic properties (such as nano-sized iron particles or other polarized nanomaterials) integrated onto the implantable biosensing platform; a metal frame used to hermetically seal the implantable unit; the external unit contains magnets to lock together with the magnets on implantable unit via magnetic attraction; the external unit contains magnetic sensors and associated circuitry which detect the magnetic field emitted from the magnetic materials when in close proximity to the implant; the external unit is equipped with audio and/or visual feedback components used to alert the user to improve alignment between the communicator and implant; the external unit is equipped with powering and wireless data receiver components which align with the power receiving and data communication components on the implantable unit.

In accordance with still yet another embodiment of the invention, a device as described herein is provided, where the proximity communicator is equipped with electromagnets; the electromagnets get energized as the magnetic field emitted from the magnetic components on the proximity communicator is sensed by the magnetic circuits located in the external unit; the external unit locks to the implantable unit as the electromagnets are energized.

In accordance with still yet another embodiment of the invention, a device as described herein is provided, where the proximity communicator is equipped with metal detection circuitry which detects the metal frame on the implantable unit.

In accordance with still yet another embodiment of the invention, a device as described herein is provided, where the proximity communicator is equipped with metal detection circuitry which detects the metal frame on the implantable unit.

In accordance with still yet another embodiment of the invention, a device as described herein is provided, where the powering component, data communication receiver and magnets are all integrated into a single device which is floating in an oil or other medium allowing the device to move freely in the −x and −y directions. The integrated powering/data receiver/magnetic component in the external unit is configured to find and align with the powering receiver and data communication component on the implantable via magnetic attraction.

In accordance with still yet another embodiment of the invention, a device as described herein is provided, where an x-y stepper motor and associated circuitry is integrated into the external proximity communicator; the x-y stepper motor is driven by positional information provided by the magnetic sensors located in the external unit; the x-y stepper motor is driven by a microcontroller and interface circuitry based on positional information provided by the magnetic sensors; the x-y-stepper motor aligns the powering and data receiver components on the proximity communicator with the power receiving and data communication components on the implantable unit.

In accordance with still yet another embodiment of the invention, a device as described herein is provided, where the electromagnets are incorporated into the proximity communicator; the electromagnets are energized once magnetic sensors indicate alignment between the proximity communicator and implantable unit; the electromagnets lock with the magnets on the implantable unit when energized.

In accordance with still yet another embodiment of the invention, a device as described herein is provided, where an array of integrated powering and data receiver components, magnetic sensors and associated interface circuitry are located in the proximity communicator; the closest magnetic sensor in the integrated powering and data receiver component in the array detects the magnets in the implantable unit; the closest integrated powering and data receiver component in the array is activated via interface circuitry.

In accordance with still yet another embodiment of the invention, a device as described herein is provided, where the electromagnets are incorporated into the proximity communicator; the electromagnets are energized once magnetic sensors indicate alignment between the proximity communicator and implantable unit; the electromagnets lock with the magnets on the implantable unit when energized.

In accordance with the present invention, the method of the invention may be implemented, wholly or partially, by a controller operating in response to a machine-readable computer program. In order to perform the prescribed functions and desired processing, as well as the computations therefore (e.g. execution control algorithm(s), the control processes prescribed herein, and the like), the controller may include, but not be limited to, a processor(s), computer(s), memory, storage, register(s), timing, interrupt(s), communication interface(s), and input/output signal interface(s), as well as combination comprising at least one of the foregoing.

Moreover, the method of the present invention may be embodied in the form of a computer or controller implemented processes. The method and/or algorithm of the invention may also be embodied in the form of computer program code containing instructions embodied in tangible media, such as floppy diskettes, CD-ROMs, hard drives, and/or any other tangible computer-readable medium, wherein when the computer program code is loaded into and executed by a computer or controller, the computer or controller becomes an apparatus for practicing the invention. The method and/or algorithm of the invention can also be embodied in the form of computer program code, for example, whether stored in a storage medium, loaded into and/or executed by a computer or controller, or transmitted over some transmission medium, such as over electrical wiring or cabling, through fiber optics, or via electromagnetic radiation, wherein when the computer program code is loaded into and executed by a computer or a controller, the computer or controller becomes an apparatus for practicing the invention. When implemented on a general-purpose microprocessor the computer program code segments may configure the microprocessor to create specific logic circuits.

It should be appreciated that while the invention has been described with reference to an exemplary embodiment, it will be understood by those skilled in the art that various changes, omissions and/or additions may be made and equivalents may be substituted for elements thereof without departing from the spirit and scope of the invention. In addition, many modifications may be made to adapt a particular situation or material to the teachings of the invention without departing from the scope thereof. Therefore, it is intended that the invention not be limited to the particular embodiment disclosed as the best mode contemplated for carrying out this invention, but that the invention will include all embodiments falling within the scope of the appended claims. Moreover, unless specifically stated any use of the terms first, second, etc. do not denote any order or importance, but rather the terms first, second, etc. are used to distinguish one element from another.

What is claimed is:

1. A method of detecting and locating the position of an implanted biosensor platform which is in communication with an external control unit, providing levels of designated analytes in subcutaneous tissue, the method comprising:
optically powering the biosensor platform via solar cells, wherein the biosensor platform is in communication wirelessly with the external control unit via optical communication using photodetector serving as receiver which receives coded instructions and transmitting analyte levels and other conditions such as power level received by solar cells using a dedicated optical transmitter,
the external unit comprises of a camera which enables an operator to position it over the implanted biosensor platform, wherein the external control unit provides at least one of a visual and audible indicator to the operator when at least one of the external control unit is located over at least a portion of the implanted biosensor platform and in response to transmission from the implanted biosensor platform;
the camera images the biosensor platform using light source in the external unit that powers the solar cells or photovoltaic devices located on one of the sub-chips in the implanted biosensor platform,
the biosensor platform comprises of sensor electrodes dedicated for measuring designated analytes such as glucose, lactate, oxygen and pH, and wherein the biosensor platform is coated with biocompatible coatings and is sealed against body fluids on all sides with the exception of the sensor electrodes,
the biosensor platform comprises of one or more Si integrated circuit sub-chips performing signal processing functions needed in the measurement of analyte levels,
surface of one or more of the sub-chips comprising of patterned magnetic regions serving as source of magnetic field,
the magnetic regions is comprised of high strength magnetic material selected from a list samarium, iron, ferrite, samaraium boron garnet,
wherein the magnetic field is detected and measured by two or more magnetic sensors located in the external control unit,
the magnetic sensors in cooperation with the microprocessor unit in the external control unit produces a 3-dimensional map of the location of the implanted biosensor platform relative to the location of the external control unit,
the external control unit comprising of an x-y stage which can be moved with respect to its frame relative to the location of the implanted biosensor platform so that a desired optimal magnetic field distribution is obtained.

2. A method of detecting, locating the position, and aligning an implanted biosensor platform relative to an external control unit, providing with reproducible measurement of levels of designated analytes in the subcutaneous tissue in which the said biosensor is implanted, the method comprising:
said external control unit comprising of one or more electromagnet coils which upon energizing by passing current of desired magnitude enables the alignment of the implanted biosensor platform,
wherein said biosensor platform is optically powered by solar cells and is in communication wirelessly with the said external control unit via optical communication using photodetector serving as receiver which receives coded instructions and transmitting analyte levels and other conditions such as power level received by solar cells using a dedicated optical transmitter,
said solar cells receives light power from at least one source located in the said external control unit,
said external unit comprises of a camera which enables the operator to position it over the implanted biosensor platform,
said biosensor platform comprises of sensor electrodes dedicated for measuring designated analytes such as glucose, lactate, oxygen and pH, and wherein said biosensor platform is coated with biocompatible coatings and is sealed against body fluids on all sides with the exception of said sensor electrodes,
said biosensor platform comprises of one or more Si integrated circuit sub-chips performing signal processing functions needed in the measurement of analyte levels,
surface of one or more said sub-chips comprising of patterned magnetic regions serving as source of magnetic field,
said magnetic regions is comprised of high strength magnetic material selected from a list samarium, iron, ferrite, samaraium boron garnet,
wherein said magnetic field is detected and measured by two or more magnetic sensors located in the said external control unit,
said magnetic sensors in cooperation with the microprocessor unit in the said external control unit produces a 3-dimensional map of the location of said implanted biosensor platform relative to the location of the external control unit,
said surface of said sub-chips in implanted biosensor platform comprising of more than one magnetic field sensors located such that said magnetic sensor minimally receives the magnetic field of the said magnetic regions,
said magnetic field sensors located on sub-chips measure the magnetic field of the electromagnetic coils housed in the external control unit, and transmit magnetic field magnitude and their distribution information using the optical transmitter of the implanted unit,
said magnetic field distribution of the electromagnetic coils is received by the photodetector of the external control unit,
said magnetic field data of the electromagnetic coils is processed by the microprocessor unit in the said external control unit, and produces a 3-dimensional map of the location of said implanted biosensor platform magnetic sensors relative to the location of the electromagnetic coils,
said external control unit comprising of an x-y stage with provision for tilting along two axes,
said x-y stage can be moved with respect to external control unit frame relative to the location of the implanted biosensor platform so that a desired optimal alignment is obtained after processing the said two sets of magnetic field distribution maps,
said implanted biosensor platform is locked with the help of magnetic field created by electromagnetic coils for repeated analyte measurements.

3. A method of detecting, locating the position, and aligning and locking in position an implanted biosensor platform relative to an external control unit, providing reproducible measurements of levels of designated analytes in the subcutaneous tissue in which the said biosensor is implanted, the method comprising:
detecting the position of the implanted biosensor platform via optical pattern recognition methods using a camera and imaging device and light sources housed in the external control unit, and optical contrast producing patterns located on the surface of sub-chips in the implanted biosensor platform,
wherein said optical contrast producing patterns are coded with 1-dimensional bar code or 2-dimensional UR code,
said coded bar code patterns are implemented using reflective metal patterns, semiconducting patterns or phosphor material patterns which are generating reflections at the wavelength of powering source that powers the said solar cells,
said imaging device comprises from a list of devices such as a 2-dimensional charge coupled device (CCD), a 2-D metal-oxide semiconductor (MOS) imaging device,
wherein said biosensor platform is optically powered by solar cells and is in communication wirelessly with the said external control unit via optical communication using photodetector serving as receiver which receives coded instructions and transmitting analyte levels and other conditions such as power level received by solar cells using a dedicated optical transmitter,
said implanted biosensor platform
wherein precision alignment and locking of the external control unit is done by electromagnetic coils, x-y stage with 2-axis tilting and rotation features, located in the external control unit,
said electromagnetic coils function in cooperation with magnetic field sensors and magnetic regions located on the sub-chip surface of the implanted unit,
said external control unit comprising of one or more electromagnet coils which upon energizing by passing current of desired magnitude enables the alignment of the implanted biosensor platform,
said optical pattern detection and alignment and magnetic locking is done in cooperation with said external control unit microprocessor and its algorithms.
